(19) United States
(12) Patent Application Publication
(10) Pub. No.: US 2026/0183105 A1
(43) Pub. Date: Jul. 2, 2026

(54) Prosthetic Valves Including Leaflets Secured Within A Frame And Methods for Leaflet Attachment

(71) Applicant: Edwards Lifesciences Corporation, Irvine, CA (US)

(72) Inventors: Roy Shitrit, Zichron Yaakov (IL); Tamir S. Levi, Zikhron Yaakov (IL); Michael Bukin, Pardes Hanna (IL)

(73) Assignee: Edwards Lifesciences Corporation, Irvine, CA (US)

(21) Appl. No.: 19/549,549

(22) Filed: Feb. 25, 2026

Related U.S. Application Data
(63) Continuation of application No. PCT/US2024/046161, filed on Sep. 11, 2024.
(60) Provisional application No. 63/538,157, filed on Sep. 13, 2023.

(51) Int. Cl. A61F 2/24

(52) U.S. Cl. CPC A61F 2/2418; A61F 2/2415; A61F 2220/0075; A61F 2230/0054; A61F 2240/001

(57) ABSTRACT

Prosthetic heart valves including a plurality of leaflets coupled to the frame and methods for leaflet attachment. The leaflets are disposed within the frame and configured to regulate the flow of blood through the frame, each leaflet comprising a cusp edge portion having a first central (apex) region and second and third side regions on opposing sides of the first region. The first region is folded and forms an inflow end of the leaflet when coupled to the frame. The side regions are coupled to the frame in an unfolded state. The leaflets can be coupled to the frame via a connecting suture line or an inner skirt.

### CROSS-REFERENCE TO RELATED APPLICATIONS

This application is a continuation of PCT Application No. PCT/US2024/046161, filed September 11, 2024, which claims the benefit of U.S. Provisional Patent Application No. 63/538,157, filed September 13, 2023, the entire contents of each of which are incorporated herein by reference.

### FIELD

The present disclosure relates to prosthetic heart valves including one or more prosthetic leaflets, and methods for securing the prosthetic leaflets to a frame and/or a skirt of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, transcatheter heart valves comprise a radially expandable and compressible cylindrical metal frame and prosthetic leaflets disposed within the frame. In some examples, the prosthetic leaflets are coupled to an inside the frame via an inner skirt. In such configurations, cusp edge portions of the leaflets can be sutured to the fabric of the inner skirt and the inner skirt can then be sutured to an inside of the frame. In some examples, the cusp edge portions of the leaflets can be sutured directly to the frame. Commissures can be formed by connecting pairs of commissure tabs of adjacent leaflets to each other and to commissure windows formed in the frame.

### SUMMARY

Described herein are prosthetic heart valves, delivery apparatus, methods for assembling prosthetic heart valves and methods for implanting prosthetic heart valves. The disclosed prosthetic heart valves and methods can, for example, limit or prevent tissue overgrowth and/or ingrowth at a cusp edge of a prosthetic leaflet of an implanted prosthetic valve by attaching a portion of the cusp edge (for example, an apex portion) in a folded configuration, while other portions of the cusp edge are coupled in an unfolded (non-folded) configuration. Such prosthetic heart valves and methods can, for example, minimize effect of the folded leaflet configuration on pressure gradients across the prosthetic valve. As such, the devices and methods disclosed herein can, among other things, overcome one or more of the deficiencies of typical prosthetic heart valves (discussed below).

A prosthetic heart valve can comprise a frame and a valve structure disposed within the frame. In addition to these components, a prosthetic heart valve can further comprise one or more of the components disclosed herein.

In some examples, a valve structure can comprise a plurality of leaflets.

In some examples, a leaflet can comprise a first region spanning a center of the cusp edge portion and a second region and a third region on opposite sides of the first region, wherein the first region is coupled to an inner surface of the frame in a folded state.

In some examples, second and third regions on opposing side of the first region are coupled to the inner surface of the frame in an unfolded state.

In some examples, first, second, and third regions of the cusp edge portion can be coupled to the inner surface of the frame with a plurality of sutures.

In some examples, a leaflet can a connecting suture line along the first, second, and third regions of the cusp edge portion.

In some examples, the plurality of sutures can extend around the connecting suture line and adjacent struts of the frame for coupling of the first, second, and third regions of the cusp edge portion to the inner surface of the frame.

In some examples, a prosthetic valve can further include an inner skirt.

In some examples, the plurality of sutures can extend through the inner skirt for coupling of the first, second, and third regions of the cusp edge portion to the inner surface of the frame.

In some examples, the second region can extend between a first end of the first region and a first commissure tab of the leaflet.

In some examples, the third region can extend between a second end of the first region and a second commissure tab.

In some examples, the cusp edge portion can include at least one of an indentation or a slit adjacent each of the first and seconds end of the first region of the cusp edge portion.

In some examples, an inflow end of a leaflet coupled to the frame is formed by the first region of the cusp edge portion in the folded state.

In some examples, an inflow end of the coupled leaflet can have one of a U-shape or V-shape apex.

In some examples, the first region of the cusp edge portion can be folded toward an outflow free edge of the leaflet.

In some examples, a leaflet can comprise a first surface and a second opposing surface, where a majority of the first surface is oriented toward an interior of the prosthetic heart valve and a majority of the second surface is oriented toward the frame.

In some examples, a portion the first surface in the first region of the cusp edge portion can be oriented toward the frame.

In some examples, a portion of the second surface in the first region of the cusp edge portion can be oriented toward the interior of the prosthetic heart valve.

In some examples, portions of the second surface in the second and third regions of the cusp edge portion can be oriented toward the frame.

In some examples, portions of the first surface in the second and third regions of the cusp edge portion can be oriented toward the interior of the prosthetic heart valve.

In some examples, a leaflet comprises an outflow free edge and a cusp edge portion, and wherein an apex region of the cusp edge portion can be coupled to the frame in a folded configuration.

In some examples, other regions of the cusp edge portion can be coupled to the frame in a non-folded configuration.

In some examples, a prosthetic heart valve comprises one or more of the components recited in Examples 1-22 below.

In some examples, a method of assembling a prosthetic heart valve can include folding an apex region of a cusp edge portion of the respective leaflet while maintaining side regions of the cusp edge portion on opposing sides of the apex region in an unfolded state.

In some examples, a method of assembling a prosthetic heart valve can include attaching the folded apex region of the cusp edge portion to an inner surface of the frame.

In some examples, a method of assembling a prosthetic heart valve can include attaching the unfolded side regions of the cusp edge portion to an inner surface of the frame.

In some examples, a method of assembling a prosthetic heart valve comprises one or more of the features recited in Examples 23-25 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a plan view of an exemplary leaflet for a prosthetic heart valve, showing a first (central) region of a cusp edge of the leaflet in an unfolded state.

FIG. 3 is a plan view of the leaflet of FIG. 2 showing the first region of the cusp edge of the leaflet in a folded configuration.

FIG. 4A is a plan view of a leaflet for a prosthetic valve, according to another example.

FIG. 4B is a perspective view of an inflow end portion of a prosthetic heart valve including a leaflet of the type shown in FIG. 4A coupled to the frame with the first (center) region of the cusp edge of the leaflet in a folded configuration.

FIG. 5 is a schematic of a cross-sectional view of a folded portion of a first cusp edge region of a leaflet connected to a frame using a connecting suture line.

FIG. 6 is a schematic of a portion of the connecting suture line of FIG. 5 and a whip stitch connected to the connecting suture line for connecting the leaflet to the frame.

FIG. 7 is another schematic showing the connecting suture wrapped around a strut of the frame and around a portion of the connecting suture line of the leaflet.

FIG. 8 is a schematic of a cross-sectional view of an unfolded portion of a second or third cusp edge region of a leaflet connected to a frame using a connecting suture line.

FIG. 9 is a schematic of a cross-sectional view of a folded portion of a first cusp edge region of a leaflet connected to a frame using an inner skirt.

FIG. 10 is a schematic of a cross-sectional view of an unfolded portion of a second or third cusp edge region of a leaflet connected to a frame using an inner skirt.

FIG. 11 is a side view of an exemplary delivery apparatus configured to deliver and implant a radially expandable prosthetic heart valve at an implantation site.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, a prosthetic valve comprises a valve structure including prosthetic leaflets (also referred to as a “leaflet assembly”) disposed within and coupled to an annular frame. In some examples, cusp edge portions of the leaflets can be sutured to an inner skirt and the inner skirt can then be sutured to an inside of the frame and/or the cusp edge portions can be sutured directly to the frame. The leaflets normally each have a V-shaped or U-shaped configuration, where an apex of the V-shape or U-shape is oriented toward in an inflow end of the prosthetic valve and an opposing edge (also referred to as a “free edge”) is oriented toward an outflow end of the prosthetic valve. Cusp edges disposed proximate to the inflow end of the prosthetic valve (for example, the apex portions of V-shaped or U-shaped leaflets) may experience tissue overgrowth and/or ingrowth when the prosthetic valve is implanted in a patient, such as at, for example, a native heart valve.

In some examples, the cusp edges of the leaflets can be folded inward to protect the cusp edges from tissue ingrowth and overgrowth. However, the folding of the cusp edges can cause the leaflet to be offset inwardly toward a longitudinal axis of the valve. Such offset may undesirably increase a pressure gradient across the implanted prosthetic valve, which can affect blood flow across the prosthetic valve.

The prosthetic valves and valve structures disclosed herein can address one or more of the foregoing issues. For example, in some examples, one or more of the leaflets in a leaflet assembly can include a first cusp edge region disposed between second and third cusp edge regions. The first cusp edge region can correspond to an apex region of a leaflet (for example, an apex portion of a V-shaped or U-shaped leaflet). The second and third cusp edge regions can be opposing side edge regions of the leaflet, which can each extend between one side of the first cusp region and one of the commissure tabs on opposing sides of the free edge.

In some examples, the first (apex) cusp edge region can be coupled to the frame in a folded state. In some examples, the second and third (side) cusp edge regions can be coupled to the frame in an unfolded state. In some examples, the first (apex) cusp edge region can include an extended portion of the leaflet that extends past or outwardly relative to the second and third (side) cusp edge regions in a pre-assembled, flattened state of the leaflet. In some examples, the extended portion can be folded inwardly toward an interior surface of the frame for attachment of the folded first (apex) cusp edge region to the frame. In some examples, the leaflet can be directly coupled to the frame. In some examples, the leaflet can be indirectly coupled to the frame via attachment to an inner skirt that is directly coupled to the frame.

Folding of the first (apex) cusp edge region for attachment to the frame can protect the cusp edge in the first region from tissue ingrowth and overgrowth when the prosthetic valve is implanted. Further, the attachment of the cusp edge in the second and third regions in an unfolded (non-folded) state can minimize inward offset of the leaflet toward the longitudinal axis of the valve, and can limit or minimize effect of the leaflet configuration on a pressure gradient across the implanted prosthetic valve.

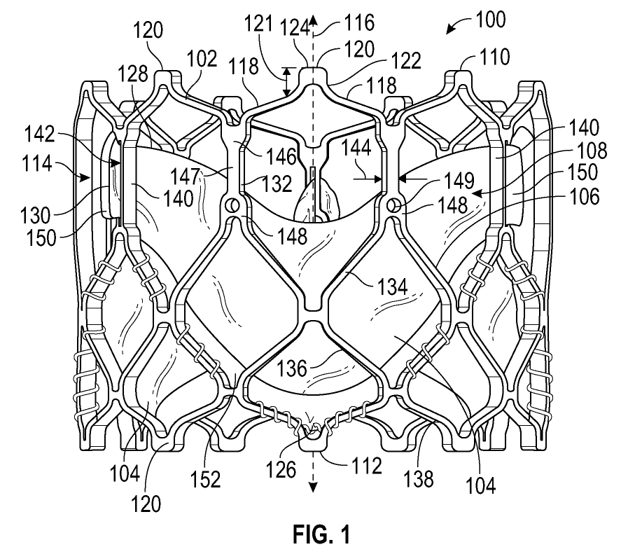

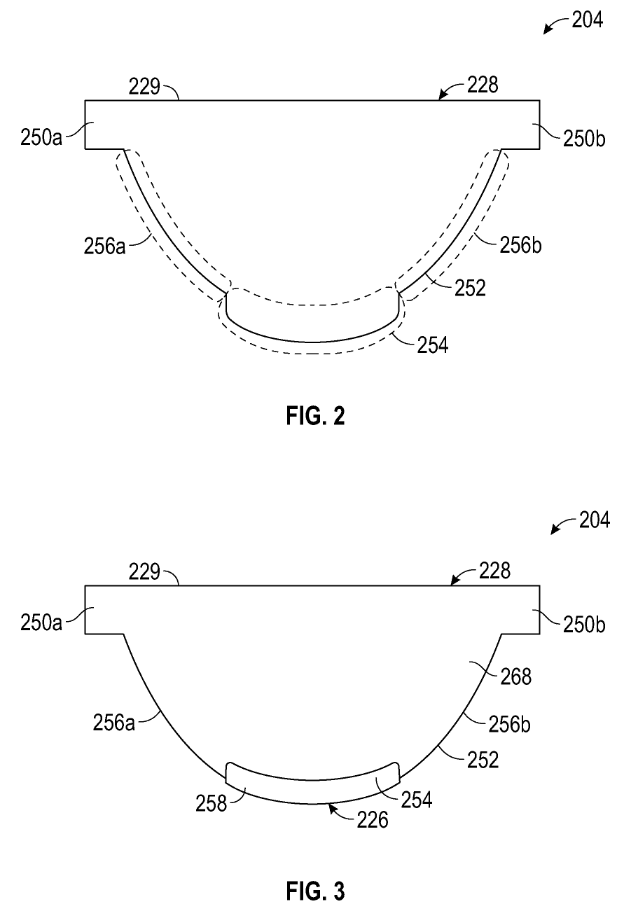

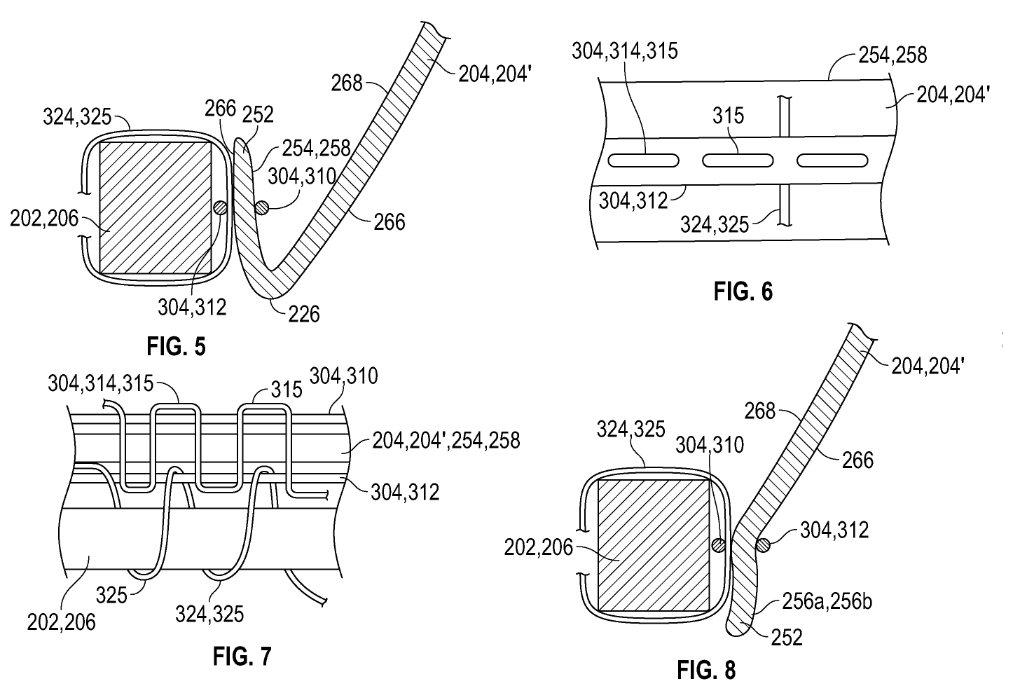

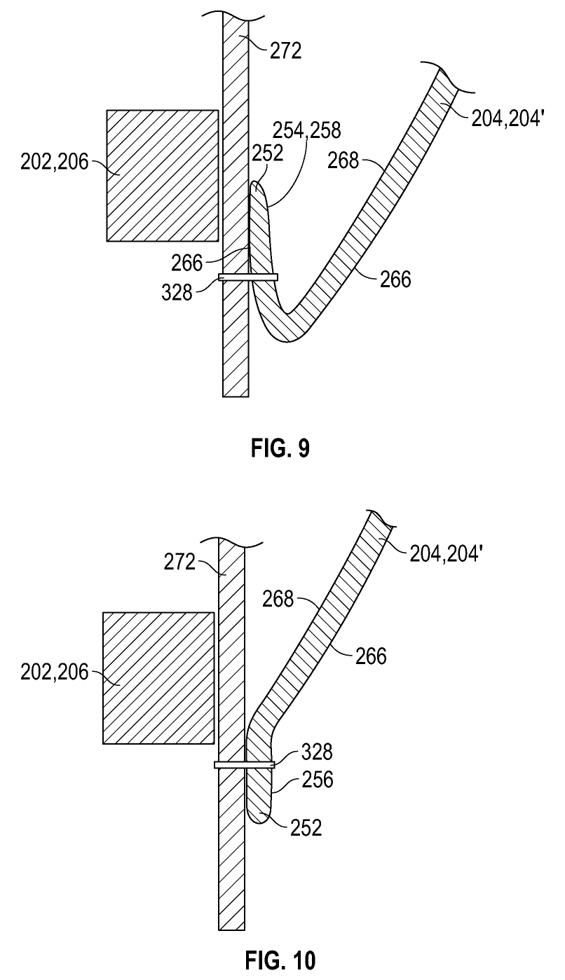

FIG. 1 illustrates an exemplary prosthetic heart valve comprising a frame, and a leaflet assembly secured on an inside of the frame. FIGS. 2-4 illustrate a leaflet including a the first (apex) cusp edge region disposed between second and third (side) cusp edge regions. FIGS. 5-8 illustrate exemplary suture configurations for attachment (coupling) of the leaflet directly to a frame. FIGS. 9-10 illustrate exemplary suture configurations for attachment of the leaflet to an inner skirt that is coupled to a frame.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later. The prosthetic heart valves can be advanced through a patient’s vasculature, such as to a native heart valve, by a delivery apparatus, such as the exemplary delivery apparatus shown in FIG. 10.

### Examples of the Disclosed Technology

FIG. 1 shows an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

As can be seen in FIG. 1, the prosthetic heart valve 100 can comprise a radially expandable and compressible annular frame 102 and a plurality of leaflets 104 secured to the frame. Each leaflet 104 can comprise opposing commissure tabs 150 disposed on opposite sides of the leaflet 104 and a cusp edge portion 152 extending between the opposing commissure tabs. The cusp edge portion 152 of each leaflet 104 can have an inflow end 126 (or edge, disposed at a central portion of the cusp edge portion 152) that is arranged at an inflow end of the prosthetic heart valve (for example, the inflow end 112 of a frame 102 of the prosthetic heart valve 100).

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 102 can comprise a plurality of interconnected struts 106 which form multiple rows of open cells 108 between an outflow end 110 and an inflow end 112 of the frame 102. In some examples, as shown in FIG. 1, the frame 102 can comprise three rows of cells 108 with a first (for example, upper in FIG. 1) row of cells 114, disposed at the outflow end 110, having cells 108 that are elongated in an axial direction (relative to a central longitudinal axis 116 of the frame 102), as compared to cells 108 in the remaining rows of cells. For example, the cells 108 of the first row of cells 114 can have a longer axial length, defined in a direction of a central longitudinal axis 116 of the frame 102, than cells 108 in the remaining rows of cells (for example, cells in the row of cells at the inflow end 112).

In some examples, as shown in FIG. 1, each row of cells 108 comprises nine cells. Thus, in such examples, the frame 102 can be referred to as a nine-cell frame.

In other examples, the frame 102 can comprise more than three rows of cells (for example, four or five) and/or more or less than nine cells per row. In some examples, the cells 108 in the first row of cells 114 may not be elongated compared to cells 108 in remaining rows of cells of the frame 102.

The interconnected struts 106 can include a plurality of angled struts 118, 134, 136, and 138 arranged in a plurality of rows of circumferentially extending rows of angled struts, with the rows being arrayed along the length of the frame between the outflow end 110 and the inflow end 112 of the frame 102. For example, the frame 102 can comprise a first row of angled struts 138 arranged end-to-end and extending circumferentially at the inflow end 112 of the frame; a second row of circumferentially extending, angled struts 136; a third row of circumferentially extending, angled struts 134; and a fourth row of circumferentially extending, angled struts 118 at the outflow end 110 of the frame 102. The fourth row of angled struts 118 can be connected to the third row of angled struts 134 by a plurality of axially extending window strut portions 140 and a plurality of axial (for example, axially extending) struts 132. The axially extending window strut portions 140 define commissure windows (for example, open windows) 142 that are spaced apart from one another around the frame 102, in a circumferential direction, and which are adapted to receive a pair of commissure tabs of a pair of adjacent leaflets 104 arranged into a commissure 130. The window strut portions 140 forming the commissure windows 142 can also be referred to herein as commissure supports of the frame 102.

One or more (for example, two, as shown in FIG. 1) axial struts 132 can be positioned between, in the circumferential direction, two commissure windows 142 formed by the window strut portions 140. Since the frame 102 can include fewer cells per row (for example, nine) and fewer axial struts 132 between each commissure window 142, as compared to known prosthetic heart valves, each cell 108 can have an increased width (in the circumferential direction), thereby providing a larger opening for blood flow and/or coronary access, as described herein.

Each axial strut 132 and each window strut portion 140 extends from a location defined by the convergence of the lower ends (for example, ends arranged inward of and farthest away from the outflow end 110) of two angled struts 118 (which can also be referred to as an upper strut junction or upper elongated strut junction) to another location defined by the convergence of the upper ends (for example, ends arranged closer to the outflow end 110) of two angled struts 134 (which can also be referred to as a lower strut junction or lower elongate strut junction). Each axial strut 132 and each window strut portion 140 forms an axial side of two adjacent cells of the first row of cells 114.

In some examples, as shown in FIG. 1, each axial strut 132 can have a width 144 that is larger than a width of the angled struts 118, 134, 136, and/or 138. As used herein, a “width” of a strut is measured between opposing locations on opposing surfaces of a strut that extend between the radially facing inner and outer surfaces of the strut (relative to the central longitudinal axis 116 of the frame 102). A “thickness” of a strut is measured between opposing locations on the radially facing inner and outer surfaces of a strut and is perpendicular to the width of the strut. In some examples, the width 144 of the axial struts 132 is 50-200%, 75-150%, or at least 100% larger than (for example, double) the width of the angled struts of the frame 102. For example, if the angled struts 118, 134, 136, and 138 are approximately 0.3 mm wide, then the width 144 of the axial struts 132 can be in a range of 0.45 mm – 0.9 mm, 0.5 mm – 0.75 mm, or at least 0.6 mm.

By providing the axial struts 132 with the width 144 that is greater than the width of other struts (for example, angled struts of the frame 102), a larger contact area is provided for when the leaflets 104 contact the wider axial struts 132 during systole. This can, for example, distribute the stress and reduce the extent to which the leaflets 104 fold over the axial struts 132 and/or extend radially outward through the cells 108, and thereby increase the long-term durability of the leaflets 104.

In some cases, the free edges at the outflow end 128 of the leaflets 104 may press against the axial struts 132 at their outflow (for example, upper) end portions 146. Accordingly, in some examples, the outflow end portions 146 of the axial struts 132 can be even wider than the width 144, which is depicted at an intermediate location of the axial strut 132 in FIG. 1, thereby providing an even larger area of contact and support for the leaflets 104.

As introduced above, since the frame 102 can have fewer cells in the circumferential direction (for example, nine in the example depicted in FIG. 1), each cell 108 can have an increased width (measured in the circumferential direction) compared to known prosthetic valves. This increased width of the cells 108 of the first row of cells 114 can enable the wider axial struts 132 to be incorporated into the frame 102, without sacrificing open space for blood flow and/or coronary access.

Commissure tabs 150 of adjacent leaflets 104 can be secured together to form commissures 130. Each commissure 130 of the prosthetic heart valve 100 comprises two commissure tabs 150 paired together, one from each of two adjacent leaflets 104, and extending through a commissure window 142 of the frame 102. Each commissure 130 can be secured to the window strut portions 140 forming the commissure window 142.

The cusp edge portion (for example, scallop edge) 152 of each leaflet 104 can be secured to the frame via one or more fasteners (for example, sutures). In some examples, as shown in FIG. 1, the cusp edge portion of each leaflet 104 can be secured directly to the struts of the frame 102 (for example, angled struts 134, 136, and 138). For example, the cusp edge portions 152 of the leaflets 104 can be sutured to the angled struts 134, 136, 138 that generally follow the contour of the cusp edge portions of the leaflets, which can be, for example, U-shaped or V-shaped.

In some examples, the cusp edge portion 152 of the leaflets 104 can be secured to an inner skirt and the inner skirt can then be secured directly to the frame 102. In some examples, an outer skirt can be connected to, arranged on, and/or coupled to an outer surface of the frame 102.

One or more of the skirts can be wholly or partly formed of any suitable biological material, synthetic material (for example, any of various polymers), or combinations thereof. In some examples, a skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, a skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, a skirt can comprise a sponge material or foam, such as polyurethane foam. In some examples, a skirt can comprise natural tissue, such as pericardium (for example, bovine pericardium, porcine pericardium, equine pericardium, or pericardium from other sources).

As shown in FIG. 1, in some examples, one or more of or each of the axial struts 132 can comprise an inflow end portion 148 (for example, inflow end portion that is closer to the inflow end than the outflow end portion 146) that is widened relative to a middle portion 147 of the axial strut 132 (which can be defined by the width 144), similar to the outflow end portion 146 (as described above). In some examples, the inflow end portion 148 of the axial strut 132 can comprise an aperture 149. The apertures 149 can be configured to receive fasteners (for example, sutures) for attaching soft components of the prosthetic heart valve 100 to the frame 102. For example, in some examples, an outer skirt can be positioned around an outer surface of the frame 102 and secured to the apertures 149.

The frame 102 can further comprise a plurality of apices 120 formed at the inflow end 112 and the outflow end 110, each apex 120 forming a junction between two angled struts 118 at the inflow end 112 or outflow end 110 and having a height 121. As such, the apices 120 are spaced apart from one another, in a circumferential direction at the inflow end 112 and the outflow end 110.

In some examples, as shown in FIG. 1, each apex 120 can have side portions 122 that curve or bend axially outward from the angled strut 118 to which it is connected and an end portion 124 that extends between the two side portions 122 of the apex 120. The side portions 122 can extend in a direction that is parallel to the central longitudinal axis 116. The end portion 124 can be relatively flat and include a surface that is disposed normal to the central longitudinal axis 116. Each apex 120 can have two bends at its end portion 124 and two bends at the side portions 122 (for example, one at the junction between each side portion 122 and angled strut 118). In this way, the apices 120 can be U-shaped. However, in other examples, the apices 120 can have another shape such as a more curved and longer apex region with a reduced height in the axial direction, such as disclosed in PCT Application Publication No. WO2022/226147, which is incorporated herein by reference.

As introduced above, a leaflet assembly of a prosthetic heart valve comprising a plurality of leaflets (for example, leaflets 104 shown in FIG. 1) can either be secured to an inner skirt which is then secured to the struts of the frame of the prosthetic heart valve or directly to the struts of the frame (without the inner skirt). An example of directly securing the leaflets 104 to the struts 106 of the frame 102 is shown in FIG. 1.

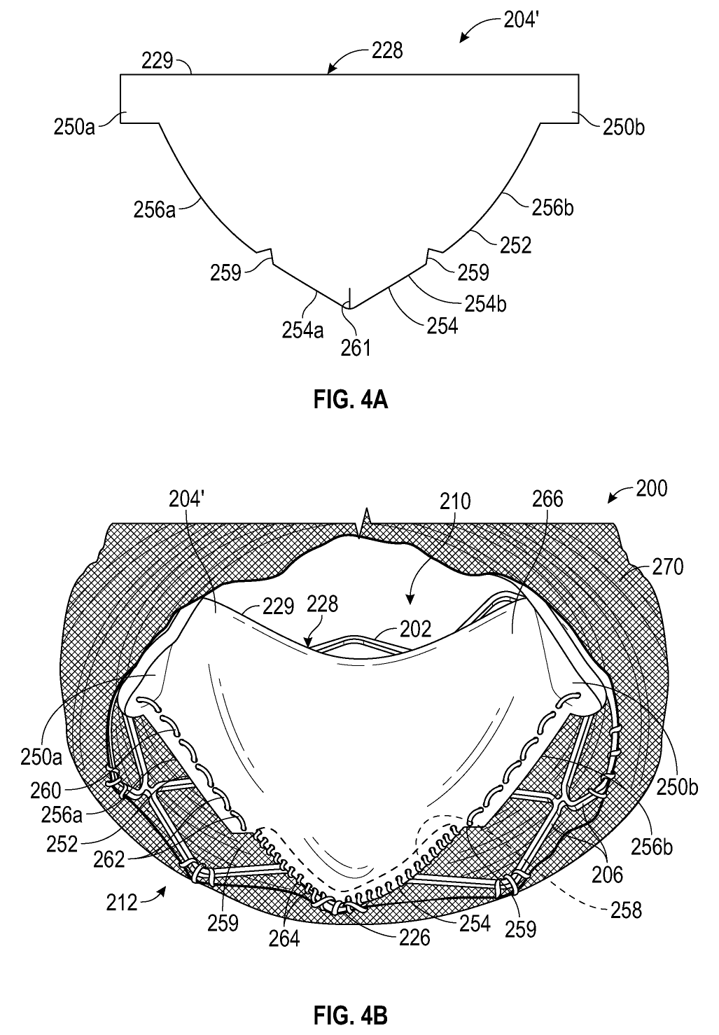

Turning to FIGS. 2 and 3, a leaflet 204, according to a first example, is shown and described. In some examples, a prosthetic heart valve comprises a leaflet assembly comprising a plurality of leaflets 204 mounted inside a frame (for example, the frame 102). FIG. 4A shows a leaflet 204’, according to another example. FIG. 4B illustrates a prosthetic valve 200 including multiple leaflets 204’ (one of which is visible in FIG. 4B), a frame 202, and an outer skirt 270. The leaflet 204’ is similar to the leaflet 204, and therefore the description of leaflet 204 applies to leaflet 204’, except for the differences discussed below. Similarly, the assembly techniques described below for assembling leaflets to the frame 202 apply to leaflet 204 and leaflet 204’.

In some examples, the prosthetic valve 200 can have one or more features of the prosthetic valve 100 described above. In some examples, the prosthetic valve 200 can have features that differ from the prosthetic valve 100. In some examples, the frame 202 of the prosthetic valve 200 can have one or more of the features of the frame 102 described above. In some examples, the frame 202 can have features that differ from the frame 102. In some examples, the outer skirt 270 can have one or more of the skirt features described above. In some examples, the outer skirt 270 can be excluded from the prosthetic valve 200. In some examples, the leaflets 204, 204’ can have one or more features of the leaflet 104 described above. In some examples, the leaflets 204, 204’ can have features that differ from the leaflet 104.

For example, similar to the leaflet 104, the leaflet 204, 204’ can include a cusp edge portion 252 extending between commissure tabs 250a, 250b disposed on opposite sides of the leaflet 204. The cusp edge portion 252 can define a scallop edge which gives the leaflet 204, 204’, for example, an overall U-shape (FIG. 3) or V-shape (FIG. 4A). A free edge 229 can extend between the commissure tabs 250a, 250b at an outflow end 228 of the leaflets 204, 204’. The commissure tabs 250a, 250b can be configured to be secured to commissure tabs of adjacent the leaflets 204, 204’ to form commissures (for example, in a similar manner to the commissures discussed above with reference to the prosthetic valve 100).

As shown in FIG. 4B, an inflow end 226 of the leaflet 204’ (or leaflet 204) can be arranged (for example, oriented toward) at an inflow end 212 of a frame 202 of the prosthetic heart valve 200, and the outflow end 228 of the leaflet 204’ (or leaflet 204) forming the free edge 229 can be arranged (for example, oriented toward) an outflow end 210 of the frame 202. The leaflet 204’ (or leaflet 204) can be disposed on an interior surface of the frame 202, while an outer skirt 270 is disposed on an exterior surface of the frame 202.

As can be seen in FIGS. 2, 3, and 4A, the cusp edge portion 252 of the leaflet 204, 204’ can include a first cusp edge region 254 disposed between second and third cusp edge regions 256a, 256b. In some examples, the first cusp edge region 254 spans a center and/or an apex of the cusp edge portion 252 of the leaflet 204, 204’. The first region 254 can be a central portion and/or an apex portion of the cusp edge portion 252. The second cusp edge region 256a can be a first side portion of the cusp edge portion 252 extending from a first end of the first (central) region 254 to the commissure tab 250a. The third cusp edge region 256b can be a second side portion of the cusp edge portion 252 extending from a second end of the first (central) region 254 to the commissure tab 250b. In some examples, the first cusp edge region 254 forms a flap that can be folded relative to the second and third cusp edge regions 2056a, 256b to form a folded layer 258.

FIG. 2 show the first cusp edge region 254 of the leaflet 204 in an unfolded configuration. As can be seen therein, in the illustrated example, the first cusp edge region 254 comprises an extension of the cusp edge portion 252 that extends past or outwardly relative to the second and third cusp edge regions 256a, 256b. In other words, in the unfolded configuration, the first cusp edge region 254 forms a protrusion along the cusp edge portion 252 such that the peripheral edges of the leaflet along the second and third cusp edge regions 256a, 256b are offset from the peripheral edge along the first cusp edge region 254.

In some examples, the cusp edge portion 252 can include indentations, notches and/or slits within the material of the leaflet on opposing sides of the first cusp edge region 254 to facilitate folding of the first cusp edge region 254 relative to the second and third cusp edge regions 256a, 256b. For example, as shown in FIG. 4A, the leaflet 204’ includes indentations 259 at opposing ends of the first cusp edge region 254. The leaflet 204’ optionally can include a slit 261 at the apex of the leaflet, which effectively divides the first cusp edge region 254 into two halves or flaps 254a, 254b, which can be folded relative to each other.

As shown in FIG. 4B, in such examples, when the first cusp edge region 254 is in its folded state, the second and third cusp edge regions 256a, 256b extend laterally outwardly relative to the first cusp edge region 254 such that stepped transitions are formed between the opposing ends of the folded first cusp edge region 254 and the second and third cusp edge regions 256a, 256b at the locations of the indentations 259. In other examples, the first cusp edge region 254 may extend past or outwardly relative to the second and third cusp edge regions 256a, 256b (similar to the leaflet 204 of FIGS. 2-3) and additionally include the indentations and/or slits 259 and/or the slit 261.

The foregoing examples of the first cusp edge region 254 can allow the leaflet 204, 204’ to maintain a U-shaped or V-shaped apex when in the folded configuration. However, in other examples, the cusp edge portion 252 can exclude an extended first cusp edge region 254 and indentations/slits. In such examples, the folded layer 258 can be formed by an apex portion of the U or V-shape, which can be folded upward to create a generally flat or straight inflow end (apex) of the leaflet when in the folded configuration. It will be appreciated that the first cusp edge region 254 can include other forms that enable folding of the leaflet material along the first cusp edge region 254, while material in the second and third cusp edge regions 256a, 256b remains unfolded (for example, while the material in the second and third cusp edge regions 256a, 256b is maintained in a generally flat configuration).

FIGS. 3 and 4B show the first cusp edge region 254 in a folded configuration to form the folded layer 258. As can be seen therein, in some examples, the folded layer 258 can be folded toward the outflow end 228 and the free edge 229 of the leaflet 204 for attachment to the frame 202. In some examples, the leaflet 204, 204’ can be coupled to the frame 202 with the folded layer 258 oriented toward an interior surface of the frame 202. In some examples, the folded layer 258 can form and define the inflow end 226 of the leaflet 204, 204’, which can be coupled at or proximate the inflow end 212 of the frame 202. In some examples, the inflow end 226 includes at least two layers of leaflet material created by the first cusp edge region 254 being in the folded state. In some examples, the first cusp edge region 254 can include more than one fold. For example, the first cusp edge region 254 can be folded over a first time, and then folded over a second time to create three layers of leaflet material at the inflow end of the attached leaflet.

In some examples, the cusp edge portion 252 in the second and third regions 256a, 256b can be coupled the frame 202 in an unfolded state. Thus, in such examples, the second and third regions 256a, 256b include a single layer of leaflet material.

In some examples, the cusp edge portion 252 of the leaflet 204 can be secured to the frame 202 via one or more fasteners (for example, sutures). In some examples, as shown in FIG. 4B, the cusp edge portion of each leaflet 204’ (or leaflet 204) can be secured directly to struts 206 of the frame 202. For example, the first cusp edge region 254 and the second and third cusp edge regions 256a, 256b of the leaflet 204’ (or leaflet 204) can be sutured along a suture line 260 to angled struts 206 that generally follow the contour of the cusp edge portion 252 with the first cusp edge region 254 in the folded configuration.

The leaflets 204, 204’ can be made of natural tissue, such as pericardial tissue (for example, bovine pericardial tissue), biocompatible synthetic materials, or various other suitable natural or synthetic materials as known in the art and described in U.S. Patent No, 6,730,118, which is incorporated by reference herein.

In some examples, the leaflet 204, 204’ includes a first surface 266 that can form at least a majority of the interior surface of the leaflet, which is oriented toward an interior of the valve 200. The first surface 266 can oppose a second surface 268 of the leaflet 204, 204’ that is oriented away from the interior of the valve 200 and toward the frame 202. In some examples, the first surface 266 is a rougher surface and the second surface 268 is a smoother surface of the leaflet material.

When the folded layer 258 is coupled to struts 206 of the frame 202, a portion of the first surface 266 located in the first region cusp edge region 254 can contact an interior surface the frame 202. As can be seen in FIG. 4B, along the first cusp edge region 254, the suture line 260 can include stitches 264 (for example, whip stitches) that that penetrate the material of the folded layer 258 and extend over (for example, around) the inflow end 226 of the leaflet 204’ (or leaflet 204) and the struts 206.

As the second and third cusp edge regions 256a, 256b are maintained in an unfolded state during attachment to struts 206 of the frame 202, a portion of the second surface 268 located in the second and third cusp edge regions 256a, 256b can contact the interior surface of the frame 202 when the leaflet 204, 204’ is coupled thereto. Further, in some examples (such as the example of FIG. 4B), the cusp edge portion 252 in the second and third cusp edge regions 256a, 256b can extend past or beyond the suture line 260. In some examples, the suture line 260 can include stitches 262 (for example, in-and-out stitches) that penetrate the material of the second and third cusp edge regions 256a, 256b and extend around the struts 206, but do not extend around the peripheral edge of the leaflet along the second and third cusp edge regions 256a, 256b.

Additional exemplary suture configurations for attachment of the leaflets 204, 204’ to the struts 206 are illustrated in FIGS. 5-8.

In some examples, the leaflet 204, 204’ can include a connecting suture line 304 (also referred to herein as “connectors”) disposed along or near the cusp edge portion 252 of the leaflet 204. The connecting suture line can be configured to enable securing of the leaflet 204, 204’ directly to a frame (for example, the frame 202 of FIG. 4B), without having to extend or thread sutures through the leaflet during attachment to the frame. In some examples, a “connecting suture line” can be defined as a line of multiple sutures that extends along the cusp edge portion on at least one side of a leaflet and that comprises at least one first suture extending along the cusp edge portion on the at least one side of the leaflet and a plurality of in-and-out stitches (for example, formed with a second suture) that extend through the at least one first suture and the cusp edge portion of the leaflet. The in-and-out stitches can secure the first suture to the leaflet. The connecting suture line can be formed on the leaflet while the leaflet is off the frame. Further, the connecting suture line can be formed (for example, stitched) manually or automatically. Additional details and configurations for a connecting suture line that can be utilized with the leaflets and prosthetic valves disclosed herein are described in PCT Application No. PCT/US2022/049666, which is incorporated by reference herein.

Returning to FIGS. 5-8, the connecting suture line 304 in the illustrated example comprises two relatively thicker sutures 310, 312 on opposite sides of the cusp edge portion 252, and a relatively thinner suture 314 that forms in-and-out-stitches 315 extending through the cusp edge portion 252 and the sutures 310, 312. A connecting suture 324, such as a force fiber or UHMPE suture, can be looped around struts 206 of the frame 202 and a portion of the connecting suture line 304 (including, for example, the thicker sutures 310, 312 and the thinner suture 314) in order to attach the leaflet 204, 204’ to the frame 202 without having to thread a needle and the connecting suture 324 through the tissue of the leaflet 204, 204’, thereby avoiding puncturing the leaflet or sewing the suture 324 through to the leaflet while attaching the leaflet 204, 204’ to the frame 202.

In some examples, the connecting suture 324 can be looped around the strut 206 and through the portions of the connecting suture line 304 between the second thicker suture 312 and the leaflet 204, 204’ (for example, between the leaflet and the segments of the one of the thicker sutures 310, 312 that extend between the crossing points/stitches 315 formed by the thinner suture 314). In this manner, the connecting suture 324 can form a plurality of whip stitches 325, each of which extends around struts 206 of the frame 202 and around the suture 312 without extending through the leaflet 204, 204’. In some examples, one or more of the whip stitches 325 in the connecting suture 324 can extend around one of the sutures 310, 312 at locations underneath the stitches 315 instead of at locations between adjacent stitches 315. In alternate examples, the connecting suture 324 can be routed through one of the thicker sutures 310, 312, or between the stitches 315 and the thicker suture 310, 312. In some examples, the whip stitches 325 of the connecting suture 324 are interlocking with at least one of the sutures 310, 312.

In some examples, the connecting suture line 304 can be used to secure the cusp edge 252 of the leaflet 204, 204’ in each of the first, second, and third cusp edge regions 254, 256a, 256b to the struts 206 of the frame 202. For example, FIG. 5 shows a cross-section illustrating attachment of the folded layer 258 to one of the struts 206 of the frame 202 via the connecting suture line 304. In another example, FIG. 8 shows a cross-section illustrating attachment of the leaflet 204, 204’ within the second and third cusp edge regions 256a, 256b via the connecting suture line 304.

As can be seen therein, the first surface 266 of the leaflet 204, 204’ within the first cusp edge region 254 can be oriented toward the strut 206 (FIG. 5), whereas, in the second and third cusp edge regions 256a, 256b, the second surface 268 of the leaflet 204, 204’ can be oriented toward the strut 206 (FIG. 8). Since the suture 312 extends over the first surface 266 and the suture 310 extends over the second surface 268 of the leaflet 204, the suture 312 can be closer to the strut 206 than the suture 310 in the first cusp edge region 254 (FIG. 5). Further, the suture 310 can be closer to the strut than the suture 312 in the second and third cusp edge regions 256a, 256b (FIG. 8). In other words, the folding of the first cusp edge region 254 can result in the orientation of the sutures 310 and 312 in the connecting suture line 304 being inverted relative to their orientation in the second and third cusp edge regions 256a, 256b.

The direct attachment of the leaflet 204, 204’ to the frame 202, as shown in FIGS. 5-8 can allow for the elimination of an inner skirt in the assembled prosthetic heart valve. The connecting suture line 304 allows for the direct attachment of the leaflet 204 to the frame 202, without puncturing the tissue of the leaflets 204, 204’ during attachment to the frame 202. Further, by utilizing thicker sutures 310 and/or 312 that have a greater thickness that other sutures, such as the thinner suture 314, the thinner suture 314 can be prevented from tearing through a tissue of the leaflets 204, 204’, thereby creating a more robust connection between the leaflet 204, 204’ and the frame 202.

As noted above, in some examples, rather than being sutured directly to a frame, the cusp edge portion 252 of the leaflet 204, 204’ can be secured to an inner skirt and the inner skirt can then be secured to the frame. Exemplary suture configurations for attachment of the leaflet 204, 204’ to an inner skirt 272 are illustrated in FIGS. 9 and 10. In some examples, the inner skirt 272 can have one or more of the skirt features discussed above with respect to the prosthetic valve 100. In some examples, the inner skirt 272 can have one or more other features.

In some examples, the inner skirt 272 can be used to secure the cusp edge 252 of the leaflet 204, 204’ in each of the first, second, and third cusp edge regions 254, 256a, 256b to the struts 206 of the frame 202. For example, FIG. 9 shows a cross-section illustrating attachment of the folded layer 258 in the first cusp edge region 254 to one of the struts 206 of the frame 202 via the inner skirt 272. In another example, FIG. 8 shows a cross-section illustrating attachment of the leaflet 204, 204’ within the second and third cusp edge regions 256a, 256b to one of the struts 206 of the frame 202 via the inner skirt 272.

As can be seen therein, the first surface 266 of the leaflet 204 within the first cusp edge region 254 can be oriented toward the strut 206 and can contact the inner skirt 272 (FIG. 9), whereas, in the second and third cusp edge regions 256a, 256b, the second surface 268 of the leaflet 204, 204’ can be oriented toward the strut 206 and contact the inner skirt 272 (FIG. 10). Sutures 328 (for example, in-and-out sutures) can extend through the material of the inner skirt 272 and through the material of the leaflet 204 in the each of the first, second, and third cusp edge regions 254, 256a, 256b for attachment of the leaflet 204, 204’ to the inner skirt. The inner skirt 272 can be connected to the frame struts 206, such as with sutures.

In some examples, the leaflet 204, 204’ and the inner skirt 272 can be assembled into a leaflet assembly prior to attachment to the frame 202. In some examples, the inner skirt 272 can first be coupled to the frame 202 (for examples, via sutures, adhesive, connectors, etc.), and the leaflet 204, 204’ can subsequently be sutured to the inner skirt 272.

In some examples, the leaflet 204, 204’ can include connecting suture line (for example, a connecting suture line similar to the connecting suture line 304) which can be utilized for attachment of the leaflet 204, 204’ to the inner skirt 272. The connecting suture line can allow for the attachment of the leaflet 204, 204’ to the inner skirt 272, without puncturing the tissue of the leaflets 204, 204’ during attachment to the inner skirt 272 and can be prevent thinner sutures from tearing through a tissue of the leaflets 204, 204’.

In some examples, the prosthetic valve 200 includes three leaflets 204, 204’ in a valvular structure (also referred to as a “leaflet assembly”). In some examples, each of the three leaflets 204, 204’ is coupled to the frame 202 in a similar manner. In some examples, one or more of the three leaflets 204, 204’ can be coupled in a different manner relative to others of the leaflets. In some examples, the prosthetic valve 200 can include additional leaflets (for example, four leaflets 204, 204’) in the valvular structure. In some examples, the prosthetic valve 200 can include fewer leaflets in the valvular structure (for example, two leaflets 204, 204’).

As noted above, the folding of the first (apex) cusp edge region 254 of the leaflets 204, 204’ for attachment to the frame 202 can protect the cusp edges 252 in the first regions 254 from tissue ingrowth and overgrowth when the prosthetic valve 200 is implanted. Further, the attachment of the cusp edge 252 in the second and third regions 256a, 256b of the leaflets 204, 204’ in the unfolded state can minimize inward offset of the leaflets 204, 204’ toward the longitudinal axis of the prosthetic valve 200, and can limit or minimize effect of the leaflets on a pressure gradient across the prosthetic valve 200 when implanted. For example, when implanted in a native heart valve, the prosthetic valve 200 can including the leaflets 204, 204’ can have a lower pressure gradient across the valve relative to a prosthetic valve including leaflets where the entirety or majority of the cusp edge is coupled to the frame in a folded configuration.

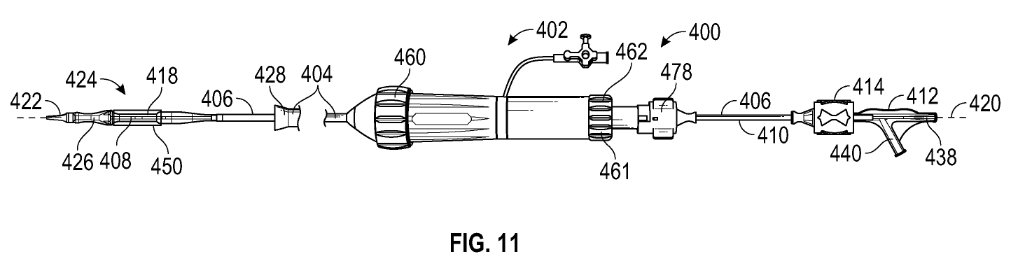

Turning to FIG. 11, an exemplary delivery apparatus 400 that can be used to implant an expandable prosthetic heart valve (for example, the prosthetic heart valve 100 of FIG. 1, the prosthetic heart valve 200 of FIG. 4B, and/or other prosthetic heart valves) is shown and described. In some examples, the delivery apparatus 400 is specifically adapted for use in introducing a prosthetic valve into a heart.

The delivery apparatus 400 in the illustrated example of FIG. 11 is a balloon catheter comprising a handle 402 and a steerable, outer shaft 404 extending distally from the handle 402. The delivery apparatus 400 can further comprise an intermediate shaft 406 (which also may be referred to as a balloon shaft) that extends proximally from the handle 402 and distally from the handle 402, the portion extending distally from the handle 402 also extending coaxially through the outer shaft 404. Additionally, the delivery apparatus 400 can further comprise an inner shaft 408 extending distally from the handle 402 coaxially through the intermediate shaft 406 and the outer shaft 404 and proximally from the handle 402 coaxially through the intermediate shaft 406.

The outer shaft 404 and the intermediate shaft 406 can be configured to translate (for example, move) longitudinally, along a central longitudinal axis 420 of the delivery apparatus 400, relative to one another to facilitate delivery and positioning of a prosthetic valve at an implantation site in a patient’s body.

The intermediate shaft 406 can include a proximal end portion 410 that extends proximally from a proximal end of the handle 402, to an adaptor 412. A rotatable knob 414 can be mounted on the proximal end portion 410 and can be configured to rotate the intermediate shaft 406 around the central longitudinal axis 420 and relative to the outer shaft 404.

The adaptor 412 can include a first port 438 configured to receive a guidewire therethrough and a second port 440 configured to receive fluid (for example, inflation fluid) from a fluid source. The second port 440 can be fluidly coupled to an inner lumen of the intermediate shaft 406.

The intermediate shaft 406 can further include a distal end portion that extends distally beyond a distal end of the outer shaft 404 when a distal end of the outer shaft 404 is positioned away from an inflatable balloon 418 of the delivery apparatus 400. A distal end portion of the inner shaft 408 can extend distally beyond the distal end portion of the intermediate shaft 406.

The balloon 418 can be coupled to the distal end portion of the intermediate shaft 406.

In some examples, a distal end of the balloon 418 can be coupled to a distal end of the delivery apparatus 400, such as to a nose cone 422 (as shown in FIG. 11), or to an alternate component at the distal end of the delivery apparatus 400 (for example, a distal shoulder). An intermediate portion of the balloon 418 can overlay a valve mounting portion 424 of a distal end portion of the delivery apparatus 400 and a distal end portion of the balloon 418 can overly a distal shoulder 426 of the delivery apparatus 400. The valve mounting portion 424 and the intermediate portion of the balloon 418 can be configured to receive a prosthetic heart valve in a radially compressed state. For example, as shown schematically in FIG. 11, a prosthetic heart valve 450 (which can be one of the prosthetic valves described herein) can be mounted around the balloon 418, at the valve mounting portion 424 of the delivery apparatus 400.

The balloon shoulder assembly, including the distal shoulder 426, is configured to maintain the prosthetic heart valve 450 (or other medical device) at a fixed position on the balloon 418 during delivery through the patient’s vasculature.

The outer shaft 404 can include a distal tip portion 428 mounted on its distal end. The outer shaft 404 and the intermediate shaft 406 can be translated axially relative to one another to position the distal tip portion 428 adjacent to a proximal end of the valve mounting portion 424, when the prosthetic valve 450 is mounted in the radially compressed state on the valve mounting portion 424 (as shown in FIG. 11) and during delivery of the prosthetic valve to the target implantation site. As such, the distal tip portion 428 can be configured to resist movement of the prosthetic valve 450 relative to the balloon 418 proximally, in the axial direction, relative to the balloon 418, when the distal tip portion 428 is arranged adjacent to a proximal side of the valve mounting portion 424.

An annular space can be defined between an outer surface of the inner shaft 408 and an inner surface of the intermediate shaft 406 and can be configured to receive fluid from a fluid source via the second port 440 of the adaptor 412. The annular space can be fluidly coupled to a fluid passageway formed between the outer surface of the distal end portion of the inner shaft 408 and an inner surface of the balloon 418. As such, fluid from the fluid source can flow to the fluid passageway from the annular space to inflate the balloon 418 and radially expand and deploy the prosthetic valve 450.

An inner lumen of the inner shaft can be configured to receive a guidewire therethrough, for navigating the distal end portion of the delivery apparatus 400 to the target implantation site.

The handle 402 can include a steering mechanism configured to adjust the curvature of the distal end portion of the delivery apparatus 400. In the illustrated example, for example, the handle 402 includes an adjustment member, such as the illustrated rotatable knob 460, which in turn is operatively coupled to the proximal end portion of a pull wire. The pull wire can extend distally from the handle 402 through the outer shaft 404 and has a distal end portion affixed to the outer shaft 404 at or near the distal end of the outer shaft 404. Rotating the knob 460 can increase or decrease the tension in the pull wire, thereby adjusting the curvature of the distal end portion of the delivery apparatus 400. Further details on steering or flex mechanisms for the delivery apparatus can be found in U.S. Patent No. 9,339,384, which is incorporated by reference herein.

The handle 402 can further include an adjustment mechanism 461 including an adjustment member, such as the illustrated rotatable knob 462, and an associated locking mechanism including another adjustment member, configured as a rotatable knob 478. The adjustment mechanism 461 is configured to adjust the axial position of the intermediate shaft 406 relative to the outer shaft 404 (for example, for fine positioning at the implantation site). Further details on the delivery apparatus 400 can be found in PCT Application No. PCT/US2021/047056, which is incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a radially expandable and compressible annular frame; and a valvular structure comprising a plurality of leaflets disposed within the frame, wherein each leaflet comprises an outflow free edge and a cusp edge portion, the cusp edge portion comprising a first region spanning a center of the cusp edge portion and a second region and a third region on opposite sides of the first region; wherein the first region is coupled to an inner surface of the frame in a folded state, and the second and third regions are coupled to the inner surface of the frame in an unfolded state; and wherein the first, second, and third regions are coupled to the inner surface of the frame with a plurality of sutures.

Example 2. The prosthetic heart valve of any example disclosed herein, particularly example 1, wherein each leaflet further comprises a first commissure tab and a second commissure tab for forming commissures with adjacent leaflets of the plurality of leaflets.

Example 3. The prosthetic heart valve of any example disclosed herein, particularly example 2, wherein the second region extends between a first end of the first region and the first commissure tab, and wherein the third region extends between a second end of the first region and the second commissure tab.

Example 4. The prosthetic heart valve of any example disclosed herein, particularly any of examples, 1-3, wherein the cusp edge portion comprises at least one of an indentation or a slit adjacent each of the first and seconds end of the first region of the cusp edge portion.

Example 5. The prosthetic heart valve of any example disclosed herein, particularly examples 1-4, wherein an inflow end of each leaflet is formed by the first region of the cusp edge portion in the folded state and has one of a U-shape or V-shape apex.

Example 6. The prosthetic heart valve of any example disclosed herein, particularly examples 1-5, wherein the first region of the cusp edge portion is folded toward the outflow free edge.

Example 7. The prosthetic heart valve of any example disclosed herein, particularly examples 1-6, wherein each leaflet comprises a connecting suture line along the first, second, and third regions of the cusp edge portion, and the plurality of sutures extend around the connecting suture line and adjacent struts of the frame for coupling of the first, second, and third regions of the cusp edge portion to the inner surface of the frame.

Example 8. The prosthetic heart valve of any example disclosed herein, particularly examples 1-7, further comprising an inner skirt coupled to the frame, wherein the plurality of sutures extend through the inner skirt for coupling of the first, second, and third regions of the cusp edge portion to the inner surface of the frame.

Example 9. The prosthetic heart valve of any example disclosed herein, particularly examples 1-8, wherein each leaflet comprises a first surface and a second opposing surface, and wherein a majority of the first surface is oriented toward an interior of the prosthetic heart valve, and wherein a majority of the second surface is oriented toward the frame.

Example 10. The prosthetic heart valve of any example disclosed herein, particularly example 9, wherein a portion of the first surface in the first region of the cusp edge portion is oriented toward the frame, and a portion of the second surface in the first region of the cusp edge portion is oriented toward the interior of the prosthetic heart valve.

Example 11. The prosthetic heart valve of any example disclosed herein, particularly examples 9 or 10, wherein portions of the second surface in the second and third regions of the cusp edge portion are oriented toward the frame, and wherein portions of the first surface in the second and third regions of the cusp edge portion are oriented toward the interior of the prosthetic heart valve.

Example 12. A prosthetic heart valve comprising: a radially expandable and compressible annular frame; and a valvular structure comprising a leaflet disposed within the frame, wherein the leaflet comprises an outflow free edge and a cusp edge portion, and wherein an apex region of the cusp edge portion is coupled to the frame in a folded configuration, and other regions of the cusp edge portion are coupled to the frame in a non-folded configuration.

Example 13. The prosthetic heart valve of any example disclosed herein, particularly example 12, wherein the cusp portion comprises at least one of an indentation or a slit adjacent each end of the apex region of the cusp edge portion.

Example 14. The prosthetic heart valve of any example disclosed herein, particularly examples 12 or 13, wherein the leaflet comprises a connecting suture line along the cusp edge portion for coupling of the cusp edge portion to an inner surface of the frame.

Example 15. The prosthetic heart valve of any example disclosed herein, particularly examples 12 or 13, further comprising an inner skirt coupled to an inner surface of the frame, wherein a plurality of sutures extend through the inner skirt for coupling of the cusp edge portion to the inner surface of the frame.

Example 16. The prosthetic heart valve of any example disclosed herein, particularly examples 12-15, wherein the leaflet comprises a first surface and a second opposing surface, and wherein a majority of the first surface is oriented toward an interior of the prosthetic valve, and wherein a majority of the second surface is oriented toward the frame.

Example 17. The prosthetic heart valve of any example disclosed herein, particularly example 16, wherein a portion of the first surface in the apex region of the cusp edge portion is oriented toward the frame, and a portion of the second surface in the apex region of the cusp edge portion is oriented toward the interior of the prosthetic heart valve.

Example 18. The prosthetic heart valve of any example disclosed herein, particularly examples 16 or 17, wherein portions of the second surface in the other regions of the cusp edge portion are oriented toward the frame, and wherein portions of the first surface in the other regions of the cusp edge portion are oriented toward the interior of the prosthetic heart valve.

Example 19. A prosthetic heart valve comprising: a radially expandable and compressible annular frame; and a valvular structure comprising a plurality of leaflets disposed within and coupled to the frame, wherein each coupled leaflet comprises a folded portion disposed at an apex region of a cusp edge portion of the leaflet, and wherein side regions of the cusp edge portion on opposing sides of the apex region are unfolded.

Example 20. The prosthetic heart valve of any example disclosed herein, particularly example 19, wherein the folded portion in the apex region comprises at least two layers of leaflet material, and wherein the opposing side regions of the cusp edge portion each comprise a single layer of leaflet material.

Example 21. The prosthetic heart valve of any example disclosed herein, particularly examples 19 or 20, wherein each leaflet comprises a connecting suture line along the apex region and the side regions of the cusp edge portion that is sutured to the frame for the attachment of the leaflets to the frame.

Example 22. The prosthetic heart valve of any example disclosed herein, particularly examples 19 or 20, further comprising an inner skirt sutured to the frame, wherein the apex region and the side regions of the cusp edge portion are sutured to the inner skirt for the attachment of the leaflets to the frame.

Example 23. A method of assembling a prosthetic heart valve comprising a radially expandable and compressible annular frame and a valvular structure comprising a plurality of leaflets, the method comprising, for each leaflet: folding an apex region of a cusp edge portion of the respective leaflet while maintaining side regions of the cusp edge portion on opposing sides of the apex region in an unfolded state; attaching the folded apex region of the cusp edge portion to an inner surface of the frame; and attaching the unfolded side regions of the cusp edge portion to an inner surface of the frame.

Example 24. The method of any example disclosed herein, particularly example 23, wherein each leaflet comprises a connecting suture line along the apex region and the side regions of the cusp edge portion, and wherein the attaching the folded apex region and the unfolded side regions comprises suturing the connecting suture line to struts of the frame.

Example 25. The method of any example disclosed herein, particularly example 23, further comprising suturing an inner skirt to struts of the frame, wherein the attaching the folded apex region and the unfolded side regions comprises suturing the folded apex region and the unfolded side regions to the inner skirt.

Example 26. A method comprising sterilizing the prosthetic heart valve of any example described herein.

Example 27. A prosthetic heart valve of any one of examples 1-22, wherein the prosthetic heart valve is sterilized.

The features described herein with regard to any example can be combined with other features described in any one or more of the other examples, unless otherwise stated. For example, any one or more of the features of one leaflet can be combined with any one or more features of another leaflet. As another example, any one or more features of one prosthetic valve can be combined with any one or more features of another prosthetic valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a radially expandable and compressible annular frame; and
a valvular structure comprising a plurality of leaflets disposed within the frame, wherein each leaflet comprises an outflow free edge and a cusp edge portion, the cusp edge portion comprising a first region spanning a center of the cusp edge portion and a second region and a third region on opposite sides of the first region;
wherein the first region is coupled to an inner surface of the frame in a folded state, and the second and third regions are coupled to the inner surface of the frame in an unfolded state; and
wherein the first, second, and third regions are coupled to the inner surface of the frame with one or more sutures.

2. The prosthetic heart valve of claim 1, wherein each leaflet further comprises a first commissure tab and a second commissure tab for forming commissures with adjacent leaflets of the plurality of leaflets.

3. The prosthetic heart valve of claim 2, wherein the second region extends between a first end of the first region and the first commissure tab, and wherein the third region extends between a second end of the first region and the second commissure tab.

4. The prosthetic heart valve of claim 1, wherein the cusp edge portion comprises at least one of an indentation or a slit adjacent each of first and second ends of the first region of the cusp edge portion.

5. The prosthetic heart valve of claim 1, wherein an inflow end of each leaflet is formed by the first region of the cusp edge portion in the folded state and has one of a U-shape or V-shape apex.

6. The prosthetic heart valve of claim 1, wherein the first region of the cusp edge portion is folded toward the outflow free edge.

7. The prosthetic heart valve of claim 1, wherein each leaflet comprises a connecting suture line along the first, second, and third regions of the cusp edge portion, and the one or more sutures comprise a plurality of stitches extending around the connecting suture line and adjacent struts of the frame for coupling of the first, second, and third regions of the cusp edge portion to the inner surface of the frame.

8. The prosthetic heart valve of claim 1, further comprising an inner skirt coupled to the frame, wherein the one or more sutures comprise a plurality of stitches extending through the inner skirt for coupling of the first, second, and third regions of the cusp edge portion to the inner surface of the frame.

9. The prosthetic heart valve of claim 1, wherein each leaflet comprises a first surface and a second opposing surface, and wherein a majority of the first surface is oriented toward an interior of the prosthetic heart valve, and wherein a majority of the second surface is oriented toward the frame.

10. The prosthetic heart valve of claim 9, wherein a portion of the first surface in the first region of the cusp edge portion is oriented toward the frame, and a portion of the second surface in the first region of the cusp edge portion is oriented toward the interior of the prosthetic heart valve.

11. The prosthetic heart valve of claim 9, wherein portions of the second surface in the second and third regions of the cusp edge portion are oriented toward the frame, and wherein portions of the first surface in the second and third regions of the cusp edge portion are oriented toward the interior of the prosthetic heart valve.

12. A prosthetic heart valve comprising:
a radially expandable and compressible annular frame; and
a valvular structure comprising a leaflet disposed within the frame, wherein the leaflet comprises an outflow free edge and a cusp edge portion, and wherein an apex region of the cusp edge portion is coupled to the frame in a folded configuration, and other regions of the cusp edge portion are coupled to the frame in a non-folded configuration.

13. The prosthetic heart valve of claim 12, wherein the cusp portion comprises at least one of an indentation or a slit adjacent each end of the apex region of the cusp edge portion.

14. The prosthetic heart valve of claim 12, wherein the leaflet comprises a connecting suture line along the cusp edge portion for coupling of the cusp edge portion to an inner surface of the frame.

15. The prosthetic heart valve of claim 12, further comprising an inner skirt coupled to an inner surface of the frame, wherein one or more sutures extend through the inner skirt for coupling of the cusp edge portion to the inner surface of the frame.

16. The prosthetic heart valve of claim 12, wherein the leaflet comprises a first surface and a second opposing surface, and wherein a majority of the first surface is oriented toward an interior of the prosthetic valve, and wherein a majority of the second surface is oriented toward the frame.

17. The prosthetic heart valve of claim 16, wherein a portion of the first surface in the apex region of the cusp edge portion is oriented toward the frame, and a portion of the second surface in the apex region of the cusp edge portion is oriented toward the interior of the prosthetic heart valve.

18. A method of assembling a prosthetic heart valve comprising a radially expandable and compressible annular frame and a valvular structure comprising a plurality of leaflets, the method comprising, for each leaflet:
folding an apex region of a cusp edge portion of the respective leaflet while maintaining side regions of the cusp edge portion on opposing sides of the apex region in an unfolded state;
attaching the folded apex region of the cusp edge portion to an inner surface of the frame; and
attaching the unfolded side regions of the cusp edge portion to an inner surface of the frame.

19. The method of claim 18, wherein each leaflet comprises a connecting suture line along the apex region and the side regions of the cusp edge portion, and wherein the attaching the folded apex region and the unfolded side regions comprises suturing the connecting suture line to struts of the frame.

20. The method of claim 18, further comprising suturing an inner skirt to struts of the frame, wherein the attaching the folded apex region and the unfolded side regions comprises suturing the folded apex region and the unfolded side regions to the inner skirt.
